(19) United States
(12) Patent Application Publication
(10) Pub. No.: US 2020/0193689 A1
(43) Pub. Date: Jun. 18, 2020

(54) INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventors: Kouichi Kato, Yokohama-shi (JP); Yohei Hashizume, Tokyo (JP)

(21) Appl. No.: 16/799,435

(22) Filed: Feb. 24, 2020

Related U.S. Application Data
(63) Continuation of application No. PCT/JP2018/030943, filed on Aug. 22, 2018.

(30) Foreign Application Priority Data
Aug. 28, 2017 (JP) 2017-163471
Aug. 28, 2017 (JP) 2017-163472

(51) Int. Cl. G06T 15/08; G06T 7/00; G06T 19/20; G16H 30/40

(52) U.S. Cl. CPC G06T 15/08; G06T 7/0012; G06T 2207/30004; G16H 30/40; G06T 2219/2016; G06T 19/20

(57) ABSTRACT

An information processing apparatus is configured to cause a display unit to display a rendered image by rendering three-dimensional medical image data in a first region set for a region to render, cause the display unit to display a moving image of the rendered images respectively associated with a plurality of the regions to render different from each other by gradually increasing the region to render from the first region to a second region in a period in which the display control unit is receiving a first instruction signal that is sent in response to an instruction from a user while the rendered image is being displayed, and terminate a process of gradually increasing the region to render when the display control unit stops receiving the first instruction signal or when the region to render reaches the second region.

### CROSS-REFERENCE TO RELATED APPLICATIONS

This application is a Continuation of International Patent Application No. PCT/JP2018/030943, filed Aug. 22, 2018, which claims the benefit of Japanese Patent Application No. 2017-163472, filed Aug. 28, 2017, and Japanese Patent Application No. 2017-163471, filed Aug. 28, 2017, both of which are hereby incorporated by reference herein in their entirety.

### TECHNICAL FIELD

The present invention relates to an information processing apparatus that causes an image based on three-dimensional medical image data to be displayed.

### BACKGROUND ART

A technology for displaying an image based on three-dimensional medical image data (volume data) generated by a medical image diagnostic apparatus (modality) is known. Japanese Patent Laid-Open No. 2013-176414 describes that three-dimensional image data (volume data) is acquired by photoacoustic imaging. Japanese Patent Laid-Open No. 2013-176414 also describes that an image is displayed by applying maximum intensity projection or volume rendering to three-dimensional image data.

However, when three-dimensional image data is rendered and displayed, information in a particular direction can be difficult to see. In this case, a user who examines a rendered image may erroneously recognize the structure of an object.

### SUMMARY OF INVENTION

The present invention provides an information processing apparatus that causes a rendered image of three-dimensional medical image data, which allows a user to easily see the structure of an object, to be displayed.

An information processing apparatus according to an embodiment of the present invention includes an image data acquisition unit configured to acquire three-dimensional medical image data, and a display control unit configured to cause a display unit to display a rendered image by rendering the three-dimensional medical image data in a region to render. The display control unit is configured to cause the display unit to display the rendered image by rendering the three-dimensional medical image data in a first region set for the region to render. The display control unit is configured to cause the display unit to display a moving image of the rendered images respectively associated with a plurality of the regions to render different from each other by gradually increasing the region to render from the first region to a second region in a period in which the display control unit is receiving a first instruction signal that is sent in response to an instruction from a user while the rendered image is being displayed. The display control unit is configured to terminate a process of gradually increasing the region to render when the display control unit stops receiving the first instruction signal or when the region to render reaches the second region.

Further features of the present invention will become apparent from the following description of exemplary embodiments with reference to the attached drawings.

### BRIEF DESCRIPTION OF DRAWINGS

FIG. 1 is a schematic diagram that shows photoacoustic image data.

FIG. 2A is a schematic diagram that shows an example of a rendered image based on the photoacoustic image data.

FIG. 2B is a schematic diagram that shows an example of a rendered image based on the photoacoustic image data.

FIG. 2C is a schematic diagram that shows an example of a rendered image based on the photoacoustic image data.

FIG. 2D is a schematic diagram that shows an example of a rendered image based on the photoacoustic image data.

FIG. 3 is a block diagram that shows an information processing system according to a first embodiment.

FIG. 4 is a block diagram that shows the specific configuration of the information processing system according to the first embodiment.

FIG. 5 is a flowchart of an information processing method according to the first embodiment.

FIG. 6 is a schematic diagram that shows a GUI according to the first embodiment.

FIG. 7 is a schematic diagram that shows a region to render according to the first embodiment.

FIG. 8A is a graph that shows the relationship between the receiving timing of an instruction signal and a region to render according to the first embodiment.

FIG. 8B is a graph that shows the relationship between the receiving timing of an instruction signal and a region to render according to the first embodiment.

FIG. 8C is a graph that shows the relationship between the receiving timing of an instruction signal and a region to render according to the first embodiment.

FIG. 8D is a graph that shows the relationship between the receiving timing of an instruction signal and a region to render according to the first embodiment.

FIG. 9A to FIG. 9F are a graph and schematic diagrams that show examples of display of a rendered image according to the first embodiment.

FIG. 10A is a graph that shows the relationship between the receiving timing of an instruction signal and a region to render according to a second embodiment.

FIG. 10B is a graph that shows the relationship between the receiving timing of an instruction signal and a region to render according to the second embodiment.

FIG. 10C is a graph that shows the relationship between the receiving timing of an instruction signal and a region to render according to the second embodiment.

FIG. 11 is a graph that shows the relationship between the receiving timing of an instruction signal and a region to render according to a third embodiment.

FIG. 12 is a schematic diagram that shows photoacoustic image data.

FIG. 13A is a schematic diagram that shows an example of a rendered image based on the photoacoustic image data.

FIG. 13B is a schematic diagram that shows an example of a rendered image based on the photoacoustic image data.

FIG. 13C is a schematic diagram that shows an example of a rendered image based on the photoacoustic image data.

FIG. 14 is a flowchart of an information processing method according to a fourth embodiment.

FIG. 15 is a schematic diagram that shows a GUI according to the fourth embodiment.

FIG. 16A is a graph that shows the relationship between the receiving timing of an instruction signal and a region to render according to the fourth embodiment.

FIG. 16B is a graph that shows the relationship between the receiving timing of an instruction signal and a region to render according to the fourth embodiment.

FIG. 16C is a graph that shows the relationship between the receiving timing of an instruction signal and a region to render according to the fourth embodiment.

FIG. 16D is a graph that shows the relationship between the receiving timing of an instruction signal and a region to render according to the fourth embodiment.

FIG. 17A to FIG. 17F are a graph and schematic diagrams that show examples of display of a rendered image according to the fourth embodiment.

FIG. 18A is a graph that shows the relationship between the receiving timing of an instruction signal and a region to render according to a fifth embodiment.

FIG. 18B is a graph that shows the relationship between the receiving timing of an instruction signal and a region to render according to the fifth embodiment.

FIG. 18C is a graph that shows the relationship between the receiving timing of an instruction signal and a region to render according to the fifth embodiment.

FIG. 19 is a graph that shows the relationship between the receiving timing of an instruction signal and a region to render according to a sixth embodiment.

### DESCRIPTION OF EMBODIMENTS

The present invention relates to an information processing apparatus that causes a rendered image based on volume data that is three-dimensional medical image data to be displayed. The present invention is applicable to medical image data that is obtained by a modality, such as a photoacoustic imaging system, an ultrasonic diagnostic system, a magnetic resonance imaging system (MRI system), an X-ray computed tomography system (X-ray CT system), and a positron emission tomography system (PET system). Specifically, the present invention can be applied to a photoacoustic imaging system that causes a rendered image based on photoacoustic image data from photoacoustic waves generated through photoirradiation to be displayed. In photoacoustic imaging, the structure of a subject to be captured cannot be completely reproduced under the influence of Limited-View unless acoustic waves can be received from directions. For this reason, the structures of blood vessels, and the like, included in photoacoustic image data can be discontinuously reconstructed. To display a structure with reduced discontinuity in the structure, the present invention can be applied when photoacoustic image data is rendered and displayed. Hereinafter, photoacoustic image data that represents the three-dimensional distribution of optical absorption coefficients will be described as an example.

Photoacoustic image data is volume data that represents the three-dimensional distribution of at least one of pieces of test sample information, such as a generated sound pressure (initial sound pressure) of photoacoustic waves, optical absorption energy density, optical absorption coefficient, the density of a substance that is a component of a biological body (such as oxygen saturation), and the like.

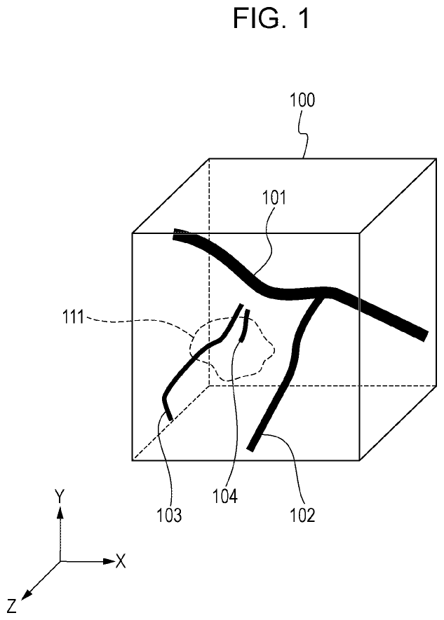

FIG. 1 is a schematic diagram of photoacoustic image data 100. The photoacoustic image data 100 shown in FIG. 1 includes image data associated with blood vessels 101, 102, 103, 104. A schematic diagram corresponding to a tumor 111 is shown by the dashed line for the sake of convenience although it is not the image data included in the photoacoustic image data 100. The blood vessel 104 is involved in the tumor 111. On the other hand, the blood vessels 101, 102, 103 are not involved in the tumor 111. Information that indicates the region of the tumor 111 may be acquired from medical image data of which a tumor is an imaging object, such as ultrasonic image data and MRI image data, based on an instruction from image processing or a user.

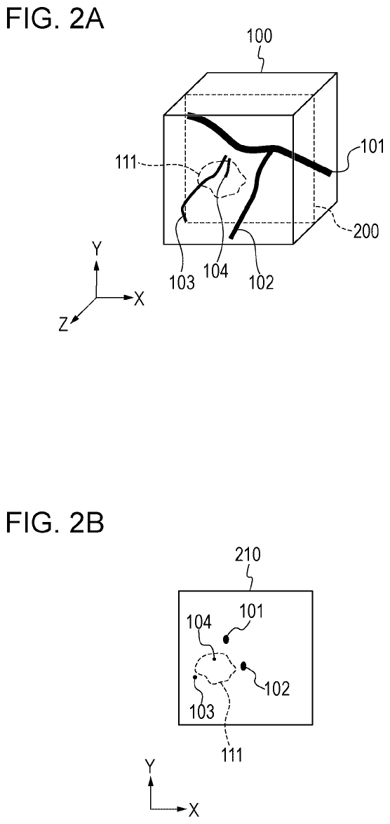

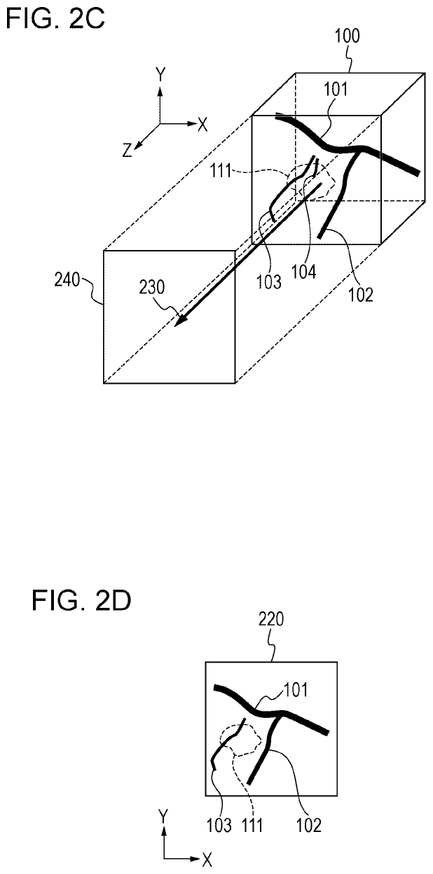

Here, assuming the case where the photoacoustic image data 100 in a cross section 200 shown in FIG. 2A is imaged. FIG. 2B is a cross-sectional image 210 of the photoacoustic image data 100 in the cross section 200. The cross-sectional image 210 shown in FIG. 2B is a rendered image obtained by rendering the photoacoustic image data 100 in the cross section 200 set for a region to render. In FIG. 2B as well, the region of the tumor 111 that intersects with the cross section 200 is shown for the sake of convenience. In the cross-sectional image 210, the images of the blood vessels 101, 102, 103, 104 that intersect with the cross section 200 are shown. When the cross-sectional image 210 is taken a look, it is understood that the blood vessel 104 is located inside the tumor 111.

However, it is difficult to see connections of blood vessels, that is, the structure of an imaging object, only by taking a look at the cross-sectional image 210. Even when cross-sectional images at some different cross-sectional positions are examined, it is difficult to observe while assuming how a blood vessel image displayed on each cross-sectional image runs with respect to the tumor 111.

Assuming the case where the photoacoustic image data 100 is projected in a Z-axis direction and displayed. Here, an example in which a projection image is displayed in maximum intensity projection will be described. FIG. 2D is a projection image 220 generated by projecting the photoacoustic image data in a line of sight direction 230 (Z-axis direction) as shown in FIG. 2C. In other words, FIG. 2D is the projection image 220 that is obtained by projecting the photoacoustic image data 100 to a projection plane 240 in maximum intensity projection. The projection image 220 shown in FIG. 2D is a rendered image obtained by rendering the photoacoustic image data 100 in all the region of the photoacoustic image data 100 set for the region to render. In FIG. 2D as well, the region of the tumor 111 that intersects with the cross section 200 is shown for the sake of convenience.

With the projection image 220 shown in FIG. 2D, connections of the blood vessels are easy to be visually identified, and the overall structure of the blood vessels is easy to understand as compared to the cross-sectional image 210 shown in FIG. 2B. Incidentally, with the projection image 220 shown in FIG. 2D, it seems that the blood vessel 103 is involved in the tumor 111; however, the blood vessel 103 is actually not involved in the tumor 111. The blood vessel 104 is shown in FIG. 2B; however, the blood vessel 104 is not shown in FIG. 2D. This is because the blood vessel 104 overlaps the blood vessel 103 in the Z-axis direction and is not shown when subjected to maximum intensity projection. When a region to render is increased, the influence of image noise can be strongly reflected in a rendered image. In this way, the visibility of an imaging object can decrease because of the influence of image noise.

As described above, it is understood that the visibility of an imaging object varies when a region to render is changed. On the other hand, an image display method that enables easy understanding of the overall structure and local structure of an imaging object without making a complicated operation is desired.

In view of this need, the inventor found an information processing method that enables a comparison among a plurality of rendered images of which the regions to render are different from one another without making a complicated operation. In other words, the inventor found an information processing method of gradually increasing a region to render when the user continues making a specific operation (for example, a user right-clicks the mouse) while a rendered image (FIG. 2B) by which the local structure of an imaging object is easy to understand is displayed. By causing a moving image of rendered images, in which the region to render gradually increases, to be displayed in this way, the user can understand a continuous change from the local structure to overall structure of an imaging object. The inventor further found that a gradual increase in the region to render is stopped when a user stops the specific operation. Thus, the user can understand a continuous change from the local structure to overall structure of an imaging object with a simple operation to whether to continue the specific operation. This mode will be described in first to third embodiments.

The inventor found an information processing method of, while the rendered image 220 of which the overall structure is easy to understand as shown in FIG. 2D is shown, a user issues an instruction and the region to render is gradually reduced in a period in which an instruction signal is being received. With this method, when a moving image of rendered images, in which the region to render gradually reduces, is displayed, the user can understand a continuous change from the total structure to the local structure with an easy operation. In addition, the inventor found that a gradual reduction in the region to render is terminated when the user stops the instruction. Thus, the user can understand a continuous change from the overall structure to local structure of an imaging object with a simple operation to whether to continue the specific operation. This mode will be described in fourth to sixth embodiments.

Incidentally, assuming a comparative example in which, when a user operates a slider bar displayed on GUI, the region to render commensurate with the position of the slider bar is determined. In such a comparative example, a user's operation is complicated when the user increases or reduces the region to render or when the user repeatedly changes the region to render. In this way, when a user's operation for changing the region to render is complicated, the efficiency of interpretation work decreases. In contrast to this, according to the embodiments of the present invention, a user is able to change the region to render by performing a simple operation to whether to continue the specific operation.

A region to render means a region (data addresses) that is subjected to rendering when three-dimensional medical image data is rendered.

Hereinafter, embodiments of the present invention will be described with reference to the attached drawings. The dimensions, materials, and shapes of components described below, the relative arrangement of them, and the like, can be changed as needed according to the configuration of an apparatus to which the invention is applied or various conditions, and the scope of the invention is not limited to the following description.

### First Embodiment

In the first embodiment of the present invention, an example of an information processing method in which, when a rendered image based on photoacoustic image data in a region to render is displayed, the region to render is gradually increased in response to an instruction from a user will be described.

Configuration of Information Processing System

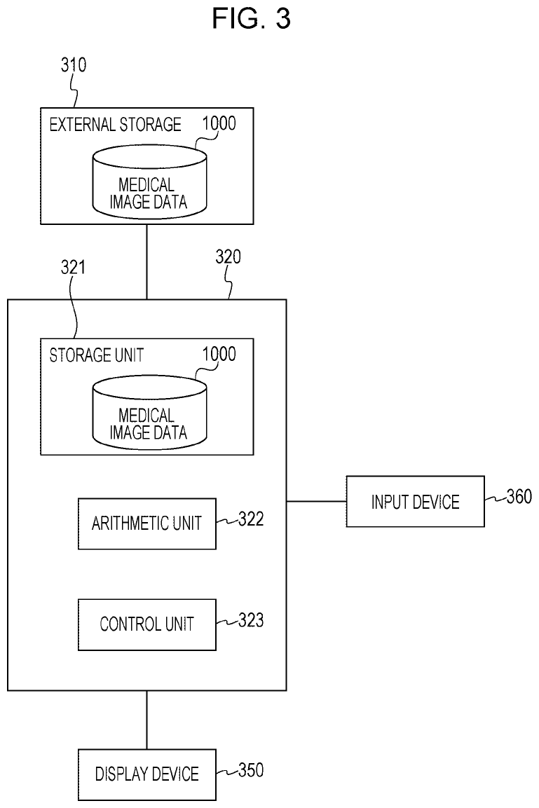

The configuration of an information processing system according to the first embodiment will be described with reference to FIG. 3. The information processing system according to the present embodiment includes an external storage 310, an information processing apparatus 320, a display device 350, and an input device 360. The information processing apparatus 320 includes a storage unit 321, an arithmetic unit 322, and a control unit 323.

### External Storage 310

The external storage 310 is disposed outside the information processing apparatus 320. Medical image data 1000 acquired by a modality is saved in the external storage 310. Medical image data 1000 of multiple image types, acquired by various modalities, may be saved in the external storage 310. The external storage 310 is made up of a recording medium, such as a server, and may be connected to a communication network, or the like. For example, a picture archiving and communication system (PACS) is used as the external storage 310 in a hospital. The external storage 310 may be provided separately from the information processing apparatus 320.

### Storage Unit 321

The storage unit 321 may be made up of a non-transitory storage medium, such as a read only memory (ROM), a magnetic disk, and a flash memory. Alternatively, the storage unit 321 may be a volatile medium, such as a random access memory (RAM). A storage medium in which a program is stored is a non-transitory storage medium. The storage unit 321 may be not only made up of a single storage medium but also made up of a plurality of storage media.

The medical image data 1000 can be acquired from the external storage 310 via the communication network, or the like, and saved in the storage unit 321.

### Arithmetic Unit 322

A unit having an image processing function as the arithmetic unit 322 can be made up of a processor, such as a CPU and a graphics processing unit (CPU), or an arithmetic circuit, such as a field programmable gate array (FPGA) chip. These units may be not only made up of a single processor or a single arithmetic circuit but also made up of a plurality of processors or a plurality of arithmetic circuits. The arithmetic unit 322 is able to read out the medical image data 1000 saved in the storage unit 321, generate a rendered image by rendering the medical image data 1000, and save the rendered image in the storage unit 321. The arithmetic unit 322 may change the details of a rendering process upon reception of an instruction signal (a signal that represents image processing conditions such as a region to render) sent from the input device 360 in response to a user's instruction.

### Control Unit 323

The control unit 323 may be made up of an arithmetic element, such as a CPU, an integrated circuit, a video random access memory (RAM). The control unit 323 is able to control the components of the information processing system. The control unit 323 may control the components of the information processing system upon reception of various instruction signals from the input device 360. The control unit 323 may control a device outside the information processing system. The control unit 323 may read out program codes stored in the storage unit 321 and control the operations of the components of the information processing system. The control unit 323 outputs a rendered image generated by the arithmetic unit 322 to the display device 350 and causes the display device 350 to display the rendered image. The control unit 323 is able to control the details of display such as the region to render for the medical image data 1000 to be displayed on the display device 350 and a period of time that is taken to change the region to render upon reception of an instruction signal sent from the input device 360 in response to a user's instruction.

The arithmetic unit 322 and the control unit 323 that generate a rendered image by rendering three-dimensional medical image data and cause the display device 350 to display the rendered image correspond to a display control unit according to embodiments of the present invention. In the present embodiment, the display control unit is made up of a plurality of units. Alternatively, the display control unit may be made up of a plurality of arithmetic elements or may be made up of a single arithmetic element.

The information processing apparatus 320 may be an exclusively designed work station. The components of the information processing apparatus 320 may be respectively made up of different hardware components. At least part of components of the information processing apparatus 320 may be made up of a single hardware component. Hardware components that make up the information processing apparatus 320 may be not accommodated in a single case.

### Display Device 350

The display device 350 that serves as a display unit is a display, such as a liquid crystal display, an organic electro luminescence (EL) display, an FED, display glasses, and a head mount display. The display device 350 displays an image based on volume data processed in the information processing apparatus 320. The display device 350 may display a GUI for manipulating an image based on volume data. The display device 350 may be provided separately from the information processing apparatus 320. At this time, the information processing apparatus 320 is able to send the medical image data 1000 to the display device 350 in a wired or wireless manner.

### Input Device 360

A mouse, a keyboard, a joystick, a touch pen, or the like, that a user can operate may be employed as the input device 360 that serves as an input unit. Alternatively, the display device 350 may be made up of a touch panel, and the display device 350 may be used as the input device 360. A microphone for inputting a voice, a camera for inputting a gesture, or the like, may be employed as the input device 360. The input device 360 may be configured to be able to input information on display of a rendered image. A numeric value may be input or input may be made by operating a slider bar as an input method. An image that is displayed on the display device 350 may be updated according to input information. Thus, a user is able to set appropriate parameters while checking an image generated based on parameters determined by user's own operation.

The input device 360 allows a user to input the conditions of a process that the arithmetic unit 322 executes or the details of display that the control unit 323 controls. Input may be made by saving a text file, or the like, that describes conditions or input may be made through a GUI displayed on the display device 350 as an input method. The input device 360 may be any device as long as the input device 360 is able to input a signal through a user's operation. A mouse, a keyboard, a touch panel, a joystick, a switch box, a microphone that receives a sound including a voice, an input device that receives a specific gesture, or the like, may be used as the input device 360. The input device 360 may be connected in a wired manner or wireless manner as long as the input device 360 is able to output an instruction signal to the information processing apparatus 320.

The components of the information processing system may be respectively made up of different devices or may be made up of an integrated single device. Alternatively, at least part of the components of the information processing system may be made up of an integrated single device.

Information that is sent or received among the components of the information processing system is exchanged in a wired manner or wireless manner.

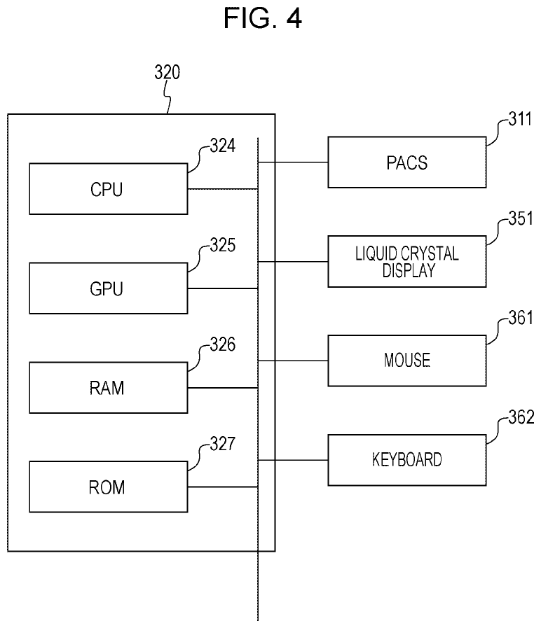

FIG. 4 is a specific configuration example of the information processing apparatus 320 according to the present embodiment. The information processing apparatus 320 according to the present embodiment is made up of a CPU 324, a GPU 325, a RAM 326, and a ROM 327. A PACS 311 that serves as the external storage 310, a liquid crystal display 351 that serves as the display device 350, and a mouse 361 and a keyboard 362 that serve as the input device 360 are connected to the information processing apparatus 320.

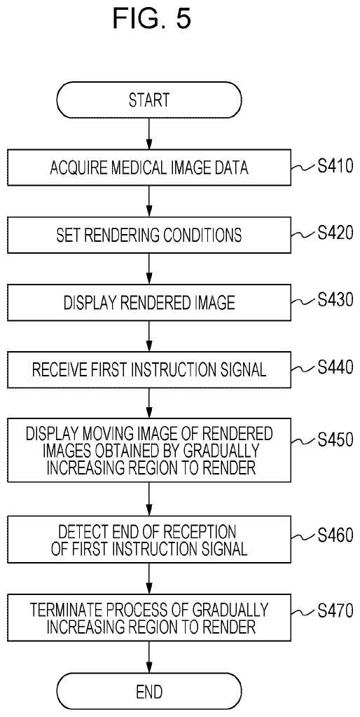

Next, a process that the information processing apparatus 320 according to the present embodiment executes will be described with reference to FIG. 5.

### S410: Step of Acquiring Medical Image Data

The control unit 323 causes the display device 350 to display a list of the pieces of medical image data 1000 saved in the external storage 310. A user selects photoacoustic image data 100 from among the list of medical image data 1000 displayed on the display device 350 with the use of the input device 360. The control unit 323 that serves as an image data acquisition unit acquires photoacoustic image data 100 that is medical image data 1000 by reading out the photoacoustic image data 100 from the external storage 310 and saves the photoacoustic image data 100 in the storage unit 321. The medical image data 1000 that serves as volume data may be a three-dimensional image made up of a plurality of cross-sectional images.

In the present embodiment, the mode in which medical image data 1000 that has been already captured by a modality is read out from the external storage 310 will be described. Alternatively, a modality may generate medical image data 1000 by starting to capture an image based on an instruction signal from the control unit 323, and the control unit 323 that serves as the image data acquisition unit may acquire the medical image data 1000 output from the modality by receiving the medical image data 1000.

### S420: Step of Setting Rendering Conditions

The control unit 323 causes the display device 350 to display a GUI for inputting rendering conditions. Specific rendering conditions include a projection direction (X, Y, or Z direction), a region to render (thickness (distance), the number of images, and reference position), a rendering method (maximum intensity projection, average intensity projection, minimum intensity projection, volume rendering, or surface rendering), and the like. A method of changing the region to render is, for example, a method of displaying an image while increasing (gradually increasing) the region to render in a stepwise manner. A period of time to change the region to render can also be set together. An image of which the region to render is increased in a stepwise manner can be displayed according to the set period of time. When the operational convenience of a user is taken into consideration, for example, a method in which an input function is assigned to a right button of the mouse is applicable. These conditions can be described in a text file, or the like, and input. Input can be made by using a GUI displayed on the display device 350.

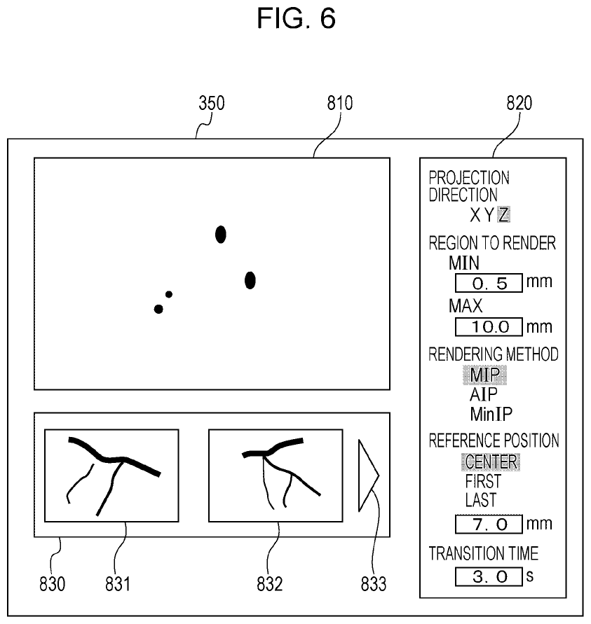

FIG. 6 is a specific example of a graphical user interface (GUI) that is displayed on the display device 350 after medical image data 1000 is selected in step S410.

A display area 810 is an area in which a rendered image of medical image data 1000 is displayed.

A display area 820 is an area in which widgets, such as a button, a list box, and a text box, for a user to input rendering conditions with the use of the input device 360 are displayed. Widgets for inputting a projection direction, a region to render, a rendering method, a reference position, and a transition time are displayed in the display area 820.

X, Y, and Z directions are displayed as choices for the projection direction, and the Z direction is selected in the drawing.

As for the region to render, a user is able to directly input the thickness (distance) L of the region to render in the projection direction in numeric value. In the drawing, a value of 0.5 mm is input as a minimum value L1 of the thickness of the region to render in the projection direction, and 10.0 mm is input as a maximum value L2. The minimum value L1 is the thickness of the region to render in the protection direction when an initial image (main image) of a rendered image is displayed. The maximum value L2 is an upper limit when a process of gradually increasing the region to render (described later) is executed. The region to render defined by the minimum value L1 corresponds to a first region. The region to render defined by the maximum value L2 corresponds to a second region. The minimum value may be preset to a value equivalent to a voxel size. To display the same area when the region to render is varied, the first region is desirably included in the second region. An initial image that is displayed first in the display area 810 may be a main image when the region to render is minimum. When imaging objects are blood vessels, the maximum value may be greater than or equal to 2 mm in order to understand connections of the blood vessels. The maximum value may be less than or equal to 10 mm in order not to perform rendering up to a redundant region. The control unit 323 may execute control not to accept input or control to provide an alert when a value not included in these ranges is input as the region to render. The minimum value may be preset to a value equivalent to a voxel size.

In the present embodiment, in order to define a region to render, an example in which the thickness (the length of one side) of a rectangular parallelepiped is input is described; however, any method is applicable as long as a region to render can be defined. For example, when a region to render is a spherical shape, the region to render may be defined by specifying the radius or diameter of a sphere. Alternatively, a region to render may be defined by specifying the number of images (the number of frames) that make up the region to render. Alternatively, a region to render may be defined by specifying the thickness of the region to render in at least one direction.

Maximum intensity projection (MIP), average intensity projection (AIP), and minimum intensity projection (MinIP) are displayed as choices for a rendering method, and MIP is selected in the drawing.

A first position, a center position, and a last position are displayed as choices for a reference position of the region to render, and the center position is selected in the drawing. The thickness of the region to render in the projection direction from an end of medical image data 1000 to the reference position can be input for the reference position, and 7.0 mm is input in the drawing. In other words, the reference position is input such that the center (reference position) of the region to render is located at a position of 7.0 mm from the end of the medical image data 1000 in the projection direction (Z-axis direction). In a process of gradually increasing the region to render (described later), the direction in which the region to render is increased varies depending on which reference position is selected. When the reference position is the first, the region to render increases from the first toward the last. When the reference position is the center, the region to render increases from the center toward both the first and the last. When the reference position is the last, the region to render increases from the last toward the first.

A user may directly input a period of time (transition time) that is taken to shift the region to render from the minimum value to the maximum value. In the drawing, 3.0 seconds is input as the condition. As for seconds to be input, as a result of the study of the inventor, several seconds are preferable to smoothly proceed with interpretation work, and preferably a period of time shorter than or equal to five seconds is selected. The control unit 323 may execute control not to accept input or control to provide an alert when a value greater than a predetermined threshold as a transition time is input. The predetermined threshold is preferably set to a value shorter than five seconds so as not to interfere with interpretation work. Furthermore, for the efficiency of interpretation work, a value shorter than three seconds is preferably set for the predetermined threshold. A value longer than one second is preferably set for the predetermined threshold so that a change of rendered images can be visually identified. Furthermore, a value longer than two seconds is preferably set for the predetermined threshold so that a change of rendered images can be further tracked and visually identified. Alternatively, not the transition time of the region to render is directly input, but the transition time may be eventually determined by inputting the amount of change per unit time in the region to render. As long as a parameter can determine the transition time of the region to render, the parameter is included in information that represents the transition time of the region to render.

For example, a user can use a mouse to select one of the choices. When a numeric value is directly input, a user can use a keyboard.

The rendering conditions are not limited to those displayed in the display area 820. Any parameters on rendering may be employed as the rendering conditions. In the present embodiment, as for a method of inputting rendering conditions as well, each rendering condition may be input by any method, such as inputting text to a text box, selecting from a list box, and depressing a button. At least one of the rendering conditions may be set in advance.

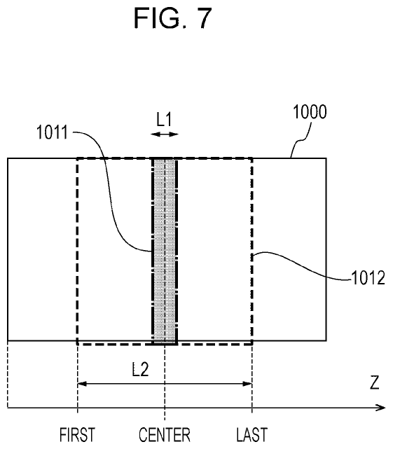

FIG. 7 is a schematic diagram that shows a region to render set for medical image data 1000. The horizontal direction of the drawing sheet corresponds to the Z-axis direction. The vertical direction of the drawing sheet corresponds to an X-axis direction or a Y-axis direction.

A region to render 1011 indicated by the alternate long and short dashed line is a region to render when the thickness in the Z-axis direction is the minimum value L1. On the other hand, a region to render 1012 indicated by the dashed line is a region to render when the thickness in the Z-axis direction is the maximum value L2. In any region to render, the center position of the region is the same. In other words, in any region to render, the center of the region to render is a reference position. In FIG. 7, the reference position (first, center, or last) of the region to render is defined with reference to the starting point of the Z-axis. Alternatively, the reference position of the region to render may be defined with reference to the end point of the Z-axis. The reference position is not limited to first, center, or last and may be set to a desired position.

A display area 830 is an area in which thumbnail images of pieces of medical image data 1000, other than the medical image data 1000 acquired in step S410, are displayed. Medical image data selected by a user from among the thumbnail images displayed in the display area 830 with the use of the input device 360 may be displayed in the display area 810. In other words, a rendered image that is displayed in the display area 810 may be updated with a rendered image of the medical image data selected from among the thumbnail images.

In the case of FIG. 6, a thumbnail image 831 is selected, and medical image data associated with the thumbnail image 831 is displayed in the display area 810. When a user operates a thumbnail image advance icon 833, thumbnail images to be displayed in the display area 830 can be sequentially changed. The thumbnail images 831, 832 that are displayed in the display area 830 may be images rendered with all the region of medical image data set for the region to render so that the overall structure can be understood in a short period of time. On the other hand, a main image that is displayed in the display area 810 may be a rendered image when the region to render is the minimum value.

In S410 as well, thumbnail images of a plurality of pieces of medical image data 1000 saved in the external storage 310 may be displayed, and medical image data that is used in the process of S430 and subsequent steps may be acquired by selecting one of the thumbnail images.

S430: Step of Displaying Rendered image

In this step, the arithmetic unit 322 generates a rendered image by rendering the medical image data 1000 based on information that indicates the rendering conditions input in step S420. Here, the region to render (first region) having a center at a position of 7.0 mm in the Z-axis direction from the end of the medical image data 1000 and having a thickness of 0.5 mm in the Z-axis direction is defined. The arithmetic unit 322 generates a rendered image by applying MIP in the region to render in the Z-axis direction set for the projection direction. The control unit 323 outputs the rendered image generated by the arithmetic unit 322 to the display device 350 and causes the display device 350 to display the rendered image. Hereinafter, the MIP image generated when the thickness of the region to render is the minimum value (when the region to render is the first region) is referred to as minimum MIP image.

### S440: Step of Receiving First Instruction Signal

The user makes an operation for changing the region to render with the use of the input device 360 when the minimum MIP image is being displayed. For example, a right-click of the mouse that serves as the input device 360 may be assigned to an operation for changing the region to render. The control unit 323 receives an instruction signal (first instruction signal) for changing the region to render, which is sent from the input device 360 in response to a user's operation. For example, when the user holds down the right mouse button, the control unit 323 is able to continue receiving the first instruction signal. In this way, a period in which the user continues the operation and the control unit 323 continues receiving the first instruction signal is referred to as a period in which the first instruction signal is being received.

S450: Step of Displaying Moving image of Rendered Images Obtained by Gradually Increasing Region to Render

The arithmetic unit 322 performs rendering while gradually increasing the region to render from the minimum value L1 to the maximum value L2, set in step S420, in a period in which the control unit 323 is receiving the first instruction signal that the control unit 323 starts receiving in step S440. The arithmetic unit 322 defines a plurality of regions to render associated with a plurality of thicknesses between the minimum value L1 and the maximum value L2, and generates rendered images respectively associated with the regions to render. The arithmetic unit 322 may sequentially generate the rendered images in order of gradually increasing the thickness of the region to render from the minimum value L1.

The control unit 323 causes the display device 350 to display the plurality of rendered images sequentially generated by the arithmetic unit 322 in order of gradually increasing the thickness of the region to render as a moving image. Thus, the user is able to check a state of gradually shifting from the minimum MIP image from which the local structure of an imaging object is easy to understand to an image from which the overall structure is easy to understand, so a continuous change from the local structure of an imaging object to the overall structure can be understood.

The control unit 323 may determine the thickness of the region to render, which reduces between frames, based on the frame rate of the moving image, the transition time, the minimum value L1, and the maximum value L2, set in S420. The frame rate may be set to greater than or equal to 10 fps to see a smooth moving image. To see a further smooth moving image, the frame rate may be set to greater than or equal to 30 fps.

### S460: Step of Detecting End of Reception of First Instruction Signal

The user terminates the operation for changing the region to render when the moving image of the rendered images is being displayed. When the user's operation is stopped, the first instruction signal from the input device 360 stops, and the control unit 323 detects the end of reception of the first instruction signal. For example, when the user stops holding down the right mouse button, the control unit 323 is able to detect the end of reception of the first instruction signal.

### S470: Step of Terminating Process of Gradually Increasing Region to Render

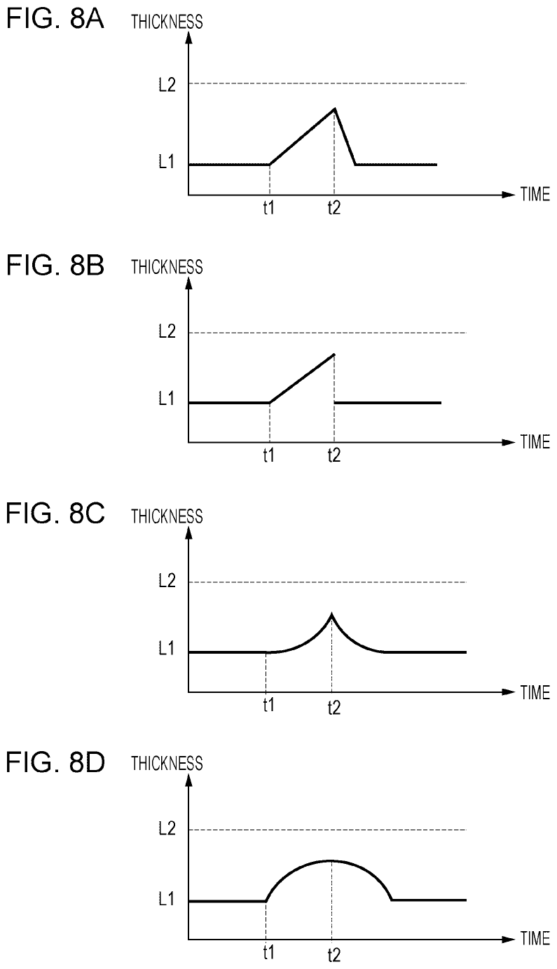

When the control unit 323 detects the end of reception of the first instruction signal, the arithmetic unit 322 terminates the process of performing rendering while gradually increasing the region to render. The rendering process and image display process after the end of reception of the first instruction signal will be described in detail in the description of graphs shown in FIG. 8A to FIG. 8D below.

FIG. 8A to FIG. 8D are graphs that show the relationship between the receiving timing of the first instruction signal and the region to render. The abscissa axis represents time, and the ordinate axis represents the thickness of the region to render. t1 is the timing at which reception of the first instruction signal starts, and t2 is the timing at which reception of the first instruction signal ends. L1 is the minimum value of the thickness of the region to render, set in step 420. L2 is the maximum value of the thickness of the region to render, set in step 420.

FIG. 8A is a graph of an example in which the amount of change per unit time in the thickness of the region to render is gradually increased at a constant rate in a period in which the first instruction signal is being received, and the thickness of the region to render is gradually reduced at a constant rate after reception of the first instruction signal ends. In this example, since a change in the thickness of the region to render is constant, the user easily intuitively understands a lapse of time and the amount of change in the region to render. In this example, after reception of the first instruction signal ends, the thickness of the region to render is gradually reduced until the region to render reaches the minimum value L1, so a continuous change from the overall structure to the local structure can be understood. Furthermore, in this example, the amount of change per unit time in the thickness of the region to render varies between when the region to render is gradually increased and when the region to render is gradually reduced. In this example, the amount of change per unit time when the region to render is gradually reduced is greater than the amount of change per unit time when the region to render is gradually increased. Thus, when the user wants to check the overall structure while checking the local structure, the user is able to check a change in structure by taking time. After checking of the overall structure completes, the screen can be quickly returned to the main image (minimum MIP image) for checking the local structure, so interpretation work can be efficiently performed.

FIG. 8B is an example in which, when reception of the first instruction signal ends, the minimum MIP image at the time when the thickness of the region to render is the minimum value is displayed. A period in which the first instruction signal is being received in FIG. 8B is similar to FIG. 8A. In this example, after checking of the overall structure completes, the focus can be quickly returned to the main image (minimum MIP image) for checking the local structure, so interpretation work can be efficiently performed.

FIG. 8C is an example in which the amount of change per unit time in the thickness of the region to render varies. In this example, when the thickness of the region to render gradually increases in a period in which the first instruction signal is being received, the amount of change per unit time gradually increases. Thus, there is no significant change in a rendered image immediately after the user starts the operation, and a change in the rendered image increases with time. Therefore, when the user wants to intensively examine a change in the local structure, the region to render may be gradually increased with this method. In this example, when the thickness of the region to render is gradually reduced after the end of reception of the first instruction signal, the amount of change per unit time gradually reduces. Therefore, when the user wants to intensively examine a change in the local structure, the region to render may be gradually reduced with this method.

Fig. SD is an example in which a mode of change in the amount of change per unit time in the thickness of the region to render differs from that of FIG. 8C. In this example, when the thickness of the region to render gradually increases in a period in which the first instruction signal is being received, the amount of change per unit time gradually reduces. On the other hand, when the thickness of the region to render is gradually reduced after the end of reception of the first instruction signal, the amount of change per unit time gradually increases. Therefore, when the user wants to intensively examine a change in the overall structure, the region to render may be gradually increased or gradually reduced in this way.

In this way, in all of the examples shown in FIG. 8A to FIG. 8D, reception of the first instruction signal stops before the thickness of the region to render reaches the maximum value L2, so the gradually increasing process terminates in response to the end of reception of the first instruction signal. When the thickness of the region to render reaches the maximum value L2 before reception of the first instruction signal ends, the gradually increasing process may be terminated when the thickness of the region to render reaches the maximum value L2. In other words, the process of gradually increasing the region to render can be terminated when reception of the first instruction signal ends or when the region to render reaches the maximum value L2 (second region).

As long as the region to render gradually increases in a period in which the first instruction signal is being received, the region to render may be changed with any method. Any combination of one of the controls over the region to render in a period in which the first instruction signal is being received, described in FIG. 8A to FIG. 8D, and one of the controls over the region to render after reception of the first instruction signal ends is applicable.

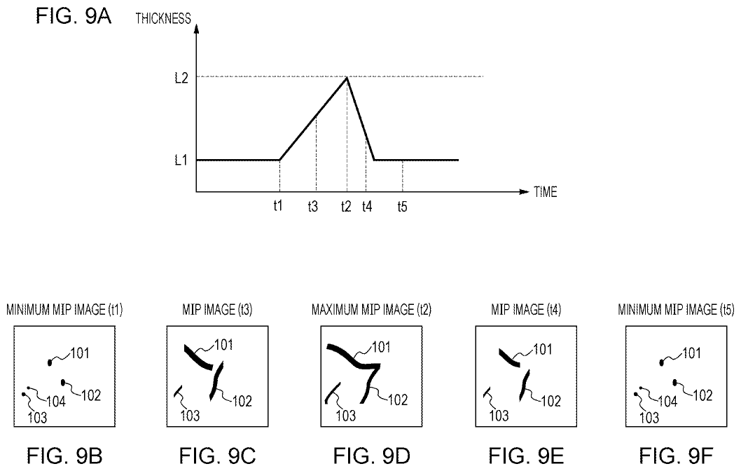

FIG. 9B to FIG. 9F show changes of a rendered image when the region to render is changed in accordance with the sequence shown in FIG. 9A.

FIG. 9B is a rendered image (minimum MIP image) when the thickness of the region to render before time t1 is the minimum value L1. FIG. 9C is a rendered image of the region to render associated with time t3 after time t1 and before the thickness of the region to render reaches the maximum value L2. FIG. 9D is a rendered image (maximum MIP image) of the region to render (maximum value L2) associated with time t2. Here, the MIP image generated at the time when the thickness of the region to render is the maximum value (when the region to render is the second region) is referred to as maximum MIP image. As shown in FIG. 9D, the blood vessels 101, 102, 103 are displayed with continuity on the maximum MIP image, and blood vessel running, or the like, can be understood. In the minimum MIP image shown in FIG. 9B, the image of the blood vessel 104 that does not appear in the MIP image shown in FIG. 9C or the MIP image shown in FIG. 9D is displayed. The blood vessel 104 overlaps the blood vessel 103 in the Z-axis direction, and is not shown when subjected to maximum intensity projection.

In this way, by gradually increasing the region to render in a period in which the first instruction signal is being received, a continuous change from the local structure to the overall structure can be understood. A user intuitively understands how a blood vessel only locally displayed is running.

FIG. 9E is a rendered image of the region to render associated with time t4 after time t2 and before the thickness of the region to render reaches the minimum value L1. FIG. 9F is a rendered image that is displayed after the thickness of the region to render reaches the minimum value L1. The rendered image shown in FIG. 9F is similar to the rendered image shown in FIG. 9B and is the minimum MIP image.

In this way, when reception of the first instruction signal ends, the region to render gradually reduces, and a continuous change from the overall structure of the imaging object to the local structure can be understood.

In the present embodiment, the image display method based on photoacoustic image data that is volume data from photoacoustic waves is described. The image display method according to the present embodiment may also be applied to volume data other than photoacoustic image data. The image display method according to the present embodiment may be applied to volume data obtained by a modality, such as an ultrasonic diagnostic system, an MRI system, an X-ray CT system, and a PET system. Particularly, the image display method according to the present embodiment may be applied to volume data including image data that represents blood vessels. Blood vessels have complex structures, and how blood vessels are running ahead cannot be estimated from a cross-sectional image. When a wide region is rendered, positional relationship among complex blood vessels cannot be understood. Therefore, the image display method according to the present embodiment may be applied to volume data including image data that represents blood vessels. For example, at least one of photoacoustic image data, magnetic resonance angiography (MRA) image data, X-ray computed tomography angiography (CTA) image data, and Doppler image data may be applied as volume data including image data that represents blood vessels.

In the present embodiment, the example in which a rendered image using medical image data of a single image type is displayed is described. Alternatively, a rendered image using medical image data of multiple image types may be displayed. For example, a rendered image generated using medical image data of an image type may be set as a base image, a rendered image may be generated with the method described in the present embodiment by using medical image data of a different image type, and the rendered image may be superimposed on the base image. In other words, a composite image obtained by combining an additional rendered image based on additional medical image data with medical image data subjected to rendering of the present embodiment may be generated and displayed. Not only a superimposed image but also a parallel image, or the like, may be employed as a composite image.

The reference position of an additional rendered image and the reference position of a rendered image subjected to rendering of the present embodiment may be associated with each other. The region to render of an additional rendered image may be set to the region at the minimum value (first region). In other words, the reference position and region to render of a minimum MIP image according to the present embodiment may be synchronized with the reference position and region to render of an additional rendered image. For example, a rendered image of MRI image data or ultrasonic image data including a tumor image may be set as a base image, rendering of the present embodiment may be applied to photoacoustic image data in which blood vessels are drawn, and a rendered image of which the region to render changes may be superimposed on the base image. Thus, since the tumor image that appears in the base image is fixed, the positional relationship between a tumor and blood vessels around the tumor can be easily understood.

An additional rendered image may be generated based on data that represents the position of an interested region, such as a tumor. Data that represents the position of an interested region may be coordinates or a function that represents the outline of the interested region or may be image data in which image values are assigned to a region in which the interested region is present. By combining a rendered image that provides such an interested region with an image rendered with the rendering method according to the present embodiment, the positional relationship between the interested region and an imaging object can be easily understood.

### Second Embodiment

In the present embodiment, the case where the region to render reaches the maximum value before reception of the first instruction signal (signal based on a gradually increasing instruction from a user) ends will be described. In the present embodiment, description will be made by using a similar apparatus to that of the first embodiment, like reference numerals denote similar components, and the detailed description thereof is omitted.

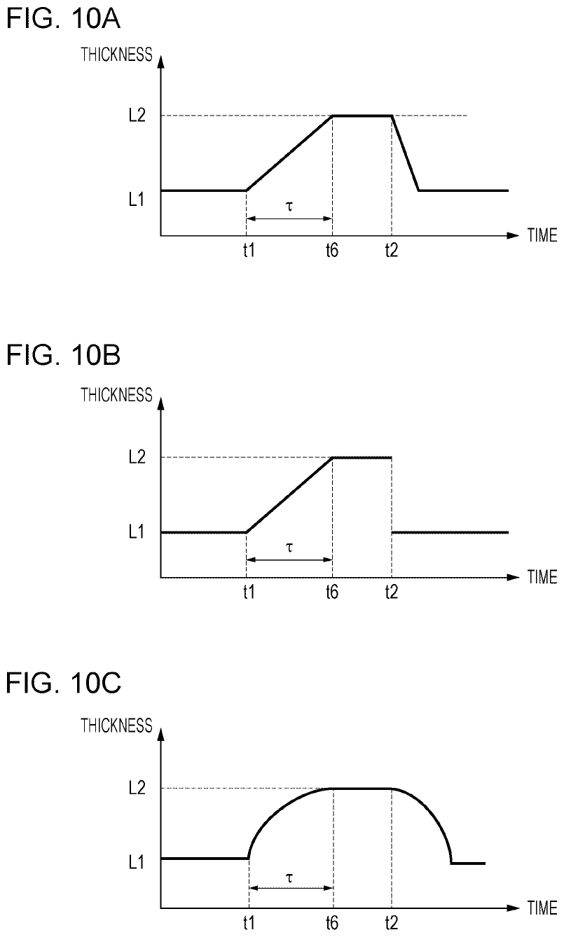

FIG. 10A to FIG. 100 are graphs that show the relationship between the receiving timing of an instruction signal and the region to render according to the present embodiment. The abscissa axis represents time, and the ordinate axis represents the thickness of the region to render. t1 is the timing at which reception of the first instruction signal starts, and t2 is the timing at which reception of the first instruction signal ends. τ is a transition time set in step S420, and t6 is the timing at which the transition time τ (predetermined period) elapses from the start of reception of the first instruction signal. L1 is the minimum value of the thickness of the region to render, set in step 420. L2 is the maximum value of the thickness of the region to render, set in step 420.

FIG. 10A to FIG. 100 are examples when a user continues an operation for gradually increasing the thickness of the region to render even after the thickness reaches the maximum value L2. In this example, the process of gradually increasing the region to render is terminated at time t6 at which the thickness of the region to render reaches the maximum value L2. In other words, the process of gradually increasing the region to render is executed such that the thickness of the region to render becomes the maximum value L2 (second region) when the transition time τ elapses from the start of reception of the first instruction signal. Subsequently, the maximum MIP image is continuously displayed after the thickness of the region to render reaches the maximum value L2 and in a period in which the first instruction signal is being received.

In FIG. 10A, when reception of the first instruction signal ends while the maximum MIP image is being displayed, the thickness of the region to render is gradually reduced to the minimum value L1. In FIG. 10B, when reception of the first instruction signal ends while the maximum MIP image is being displayed, display is switched from the maximum MIP image to the minimum MIP image. In FIG. 10C, when reception of the first instruction signal ends while the maximum MIP image is being displayed, the thickness of the region to render is gradually reduced to the minimum value L1. In FIG. 10C, the thickness of the region to render is gradually reduced to the minimum value L1 while the amount of change in the region to render is gradually increased.

In this way, while a user is continuously making an operation for checking the overall structure, the maximum NIP image is displayed. Thus, the user sufficiently understands the overall structure with a simple operation, and the user is also able to shift into an image representing the local structure with a simple operation.

In the present embodiment, the example in which display of the maximum MIP image is continued when the first instruction signal is being received at the time when the transition time r elapses from the start of reception of the first instruction signal is described. However, control over the region to render at the time when the transition time τ elapses from the start of reception of the first instruction signal is not limited to this example. For example, when the transition time τ elapses from the start of reception of the first instruction signal, the region to render may be gradually reduced to the minimum value L1 regardless of whether the first instruction signal is being received. Also, when the transition time τ elapses from the start of reception of the first instruction signal, display may be switched from the maximum MIP image to the minimum MIP image regardless of whether the first instruction signal is being received.

In the present embodiment, the example in which the region to render is gradually increased when a user operates the specific operation is described; however, the timing of starting the gradually increasing process is not limited thereto. For example, the user may perform an operation for changing the reference position of the minimum MIP image (image advance operation), and, while the minimum MIP image is being displayed through image advance, the gradually increasing process may be started when a predetermined period elapses from when the image advance operation is terminated. In other words, the information processing apparatus 320 may start the gradually increasing process when a predetermined period elapses from the end of reception of an instruction signal based on image advance operation. In this case, when the transition time τ elapses from the start of the gradually increasing process, the gradually increasing process may be terminated. An operation for scrolling the wheel of the mouse that serves as the input device 360 may be assigned to an image advance operation.

In this way, a user checks the local structure with the minimum MIP image through image advance and, when the user stops the image advance operation in the case where the user wants to check the overall structure, display can be shifted to a moving image of rendered images for understanding the overall structure. A period that is taken from the end of image advance to a transition to the gradually increasing process may be set in advance or may be designated by a user with the use of the input device 360.

### Third Embodiment

In the present embodiment, the case where a user makes an operation for gradually increasing the region to render again after reception of the first instruction signal ends or after the region to render reaches the maximum value, and in a period in which the process of gradually reducing the region to render is being executed will be described. When the user makes the operation and the information processing apparatus 320 receives a second instruction signal in response to this operation while the region to render is being gradually reduced, the process of gradually increasing the region to render is executed again. In the present embodiment, description will be made by using a similar apparatus to that of the first embodiment, like reference numerals denote similar components, and the detailed description thereof is omitted.

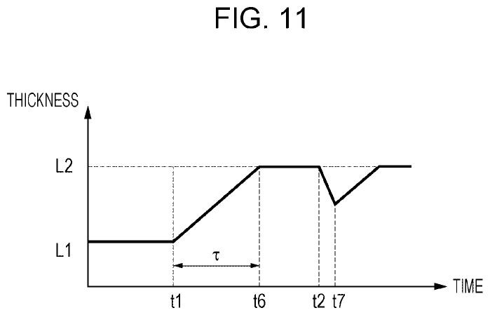

FIG. 11 is a graph that shows the relationship between the receiving timing of an instruction signal and the region to render according to the present embodiment. The abscissa axis represents time, and the ordinate axis represents the thickness of the region to render. t1 is the timing at which reception of the first instruction signal starts, and t2 is the timing at which reception of the first instruction signal ends. τ is a transition time set in step S420, and t6 is the timing at which the transition time t (predetermined period) elapses from the start of reception of the first instruction signal. t7 is the timing at which reception of the second instruction signal starts in response to an operation from the user after time t2. L1 is the minimum value of the thickness of the region to render, set in step S420. L2 is the maximum value of the thickness of the region to render, set in step S420.

FIG. 11 is an example of the case where the user makes an operation for gradually increasing the thickness again after reception of the first instruction signal ends and when the thickness of the region to render is being gradually reduced. In this example, at time t7 at which the user makes the operation again, the process of gradually reducing the region to render is terminated. Then, in a period in which the user is continuously making the operation again (that is, a period in which the second instruction signal is being received), the region to render is gradually increased to the maximum value L2. At this time, if the period that is taken to reach the maximum value L2 is determined to the transition time τ, the amount of change per unit time in the region to render varies for each operation. Therefore, the amount of change set in the first gradually increasing process may be set for the amount of change per unit time in the region to render at the time of executing the second gradually increasing process. Thus, when the gradually increasing process is executed multiple times through multiple operations, the user can check a moving image of rendered images of which the region to render gradually increases without a feeling of strangeness between operations.

When the user makes an operation for gradually increasing the thickness while the process of gradually reducing the region to render is being executed after reception of the first instruction signal ends before a lapse of the transition time τ from time t1 as well, the gradually increasing process can be repeated. In other words, when the region to render falls between the minimum value L1 and the maximum value L2 as well, the gradually increasing process and the gradually reducing process can be repeatedly executed depending on whether the first instruction signal is continuously received.

### Fourth Embodiment

Next, an information processing method in which a user issues an instruction and the region to render is gradually reduced in a period in which the instruction signal is being received will be described. An apparatus that is used in the present embodiment is similar to those of the first to third embodiments. Hereinafter, the information processing method according to the present embodiment will be described.

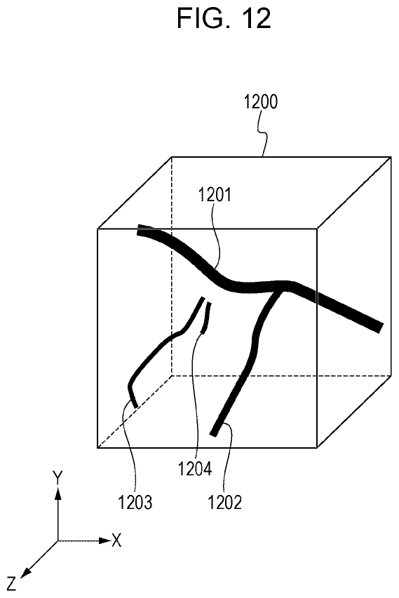

FIG. 12 is a schematic view of photoacoustic image data 1200 that represents volume data generated based on a reception signal of photoacoustic waves. The photoacoustic image data 1200 shown in FIG. 12 includes image data associated with blood vessels 1201 to 1204. As shown in FIG. 12, the blood vessels 1201 to 1204 are running in three-dimensional directions in an XYZ space.

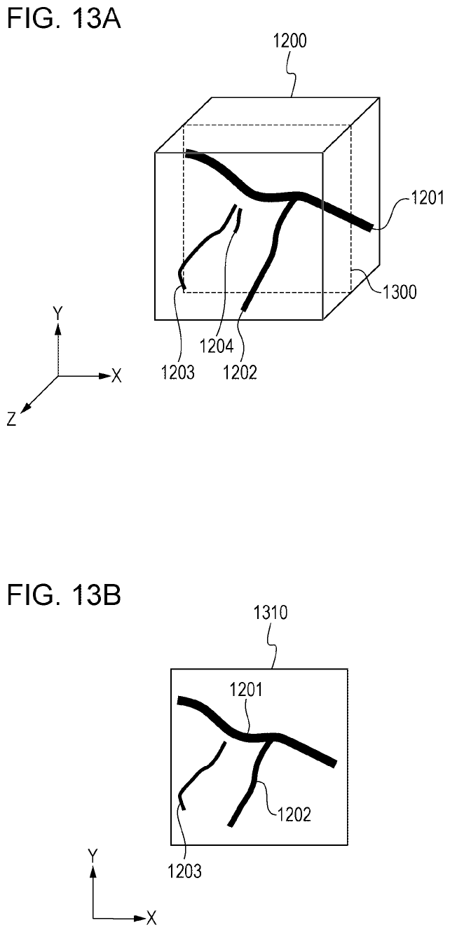

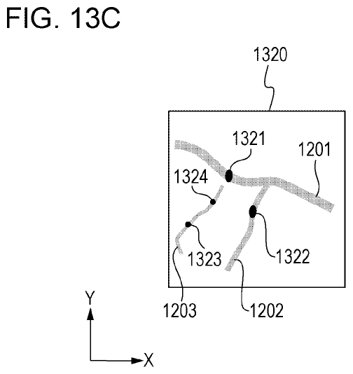

FIG. 13A is a schematic diagram of the photoacoustic image data 1200 similar to FIG. 12. FIG. 13A also shows the position of an XY cross section 1300.

FIG. 13B is a rendered image 1310 generated by rendering the photoacoustic image data 1200 in all the region in the Z-axis direction in maximum intensity protection (MIP). The images of the blood vessels 1201 to 1203 are displayed in luminance commensurate with the absorption coefficient of hemoglobin present in blood in the blood vessels.

As is apparent from the rendered image 1310 shown in FIG. 13B, continuity among the blood vessels 1201 to 1203 can be understood by rendering the photoacoustic image data 1200 in all the region. However, the blood vessel 1204 is narrower than the blood vessel 1203 and has smaller image values, so the blood vessel 1204 is hidden behind the protected image of the blood vessel 1203 and cannot be visually identified. In other words, information in a depth direction (projection direction or Z-axis direction) of the photoacoustic image data 1200 is difficult to understand as a result of rendering. Therefore, a user who sees the rendered image may erroneously recognize the positions of blood vessels. By rendering photoacoustic image data, the visibility of blood vessels can decrease because of background noise (not shown).

FIG. 13C is a cross-sectional image 1320 corresponding to the XY cross section 1300 of the photoacoustic image data 1200 of FIG. 13A. The cross-sectional image 1320 may also be regarded as a rendered image obtained by projecting the photoacoustic image data 1200 of the XY cross section 1300 in the Z-axis direction. For the sake of convenience, in order to easily understand the positional relationship among blood vessels in the cross-sectional image 1320, the rendered images of the blood vessels 1201 to 1203 in FIG. 13B are displayed in gray color in a superposition manner; however, these are not shown in the actual cross-sectional image 1320. In the cross-sectional image 1320, a blood vessel 1321 is part of the blood vessel 1201, a blood vessel 1322 is part of the blood vessel 1202, a blood vessel 1323 is part of the blood vessel 1203, and a blood vessel 1324 is part of the blood vessel 1204. The blood vessel 1204 cannot be visually identified from the rendered image 1310; however, part of the structure can be visually identified from the cross-sectional image 1320.

However, blood vessels are displayed as dots in a cross-sectional image, so continuity of blood vessels, that is, the overall structure of an imaging object, is difficult to understand. Therefore, even when a cross-sectional image is checked while the position of the cross section is changed, it is difficult to observe while estimating the running status of blood vessels.

As described above, it is understood that the visibility of an imaging object changes when the region to render is changed. On the other hand, an image display method that enables visual identification of the overall structure and local structure of an imaging object without a complicated operation is desired from users. A region to render means a region (data addresses) that is subjected to rendering when three-dimensional medical image data is rendered.

In view of this need, the inventor found an information processing method that enables a comparison among a plurality of rendered images of which the regions to render are different from one another without making a complicated operation. In other words, the inventor found an information processing method of, while the rendered image 1310 of which the overall structure is easy to understand as shown in FIG. 13B is shown, a user issues an instruction and the region to render is gradually reduced in a period in which the instruction signal is being received. With this method, when a moving image of rendered images, in which the region to render gradually reduces, is displayed, the user can understand a continuous change from the total structure to the local structure with an easy operation. In addition, the inventor found that a gradual reduction in the region to render is terminated when the user stops the instruction. Thus, the user is able to understand a change in the structure of an imaging object only by starting or stopping the instruction.

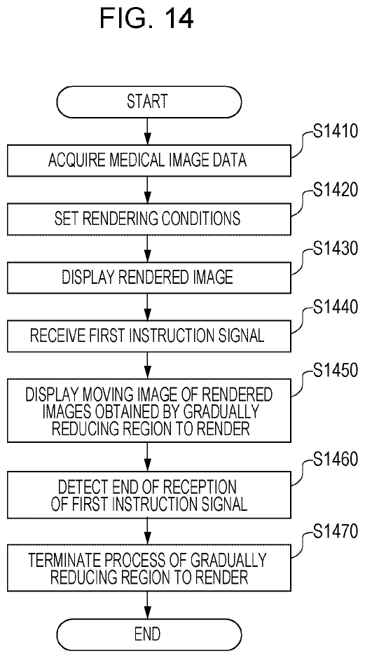

Next, a process that the information processing apparatus 320 according to the present embodiment executes will be described with reference to FIG. 14.

S1410: Step of Acquiring Medical image Data

The control unit 323 causes the display device 350 to display a list of pieces of medical image data 1000 saved in the external storage 310. A user selects photoacoustic image data 100 from among the list of medical image data 1000 displayed on the display device 350 with the use of the input device 360. The control unit 323 that serves as the image data acquisition unit acquires photoacoustic image data 100 that is medical image data 1000 by reading out the photoacoustic image data 100 from the external storage 310 and saves the photoacoustic image data 100 in the storage unit 321. The medical image data 1000 that serves as volume data may be a three-dimensional image made up of a plurality of cross-sectional images.

In the present embodiment, the mode in which medical image data 1000 that has been already captured by a modality is read out from the external storage 310 will be described. Alternatively, a modality may generate medical image data 1000 by starting to capture an image based on an instruction signal from the control unit 323, and the control unit 323 that serves as the image data acquisition unit may acquire the medical image data 1000 output from the modality by receiving the medical image data 1000.

### S1420: Step of Setting Rendering Conditions

The control unit 323 causes the display device 350 to display a GUI for inputting rendering conditions. Specific rendering conditions include a projection direction (X, Y, or Z direction), a region to render (thickness (distance), the number of images, and reference position), a rendering method (maximum intensity projection, average intensity projection, minimum intensity projection, volume rendering, or surface rendering), and the like. Examples of a method of changing a region to render include a method of displaying an image while reducing (gradually reducing) the region to render in a stepwise manner. A period of time to change the region to render can also be set together. An image of which the region to render is reduced in a stepwise manner can be displayed according to the set period of time. When the operational convenience of a user is taken into consideration, for example, a method in which an input function is assigned to a right button of the mouse is applicable. These conditions can be described in a text file, or the like, and input. Input can be made by using a GUI displayed on the display device 350.

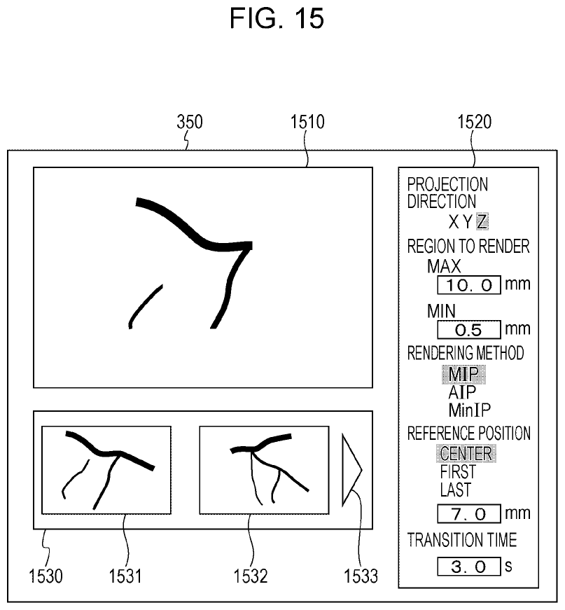

FIG. 15 is a specific example of a graphical user interface (GUI) that is displayed on the display device 350 after medical image data 1000 is selected in step S1410.

A display area 1510 is an area in which a rendered image of medical image data 1000 is displayed.

A display area 1520 is an area in which widgets, such as a button, a list box, and a text box, for a user to input rendering conditions with the use of the input device 360 are displayed. Widgets for inputting a projection direction, a region to render, a rendering method, a reference position, and a transition time are displayed in the display area 1520.

X, Y, and Z directions are displayed as choices for the projection direction, and the Z direction is selected in the drawing.

As for the region to render, a user is able to directly input the thickness (distance) L of the region to render in the projection direction in numeric value. In the drawing, a value of 10.0 mm is input as the maximum value L2 of the thickness of the region to render in the projection direction, and 0.5 mm is input as the minimum value Li. The maximum value L2 is the thickness of the region to render in the projection direction when an initial image (main image) of a rendered image is displayed. The minimum value L1 is a lower limit when a process of gradually reducing the region to render (described later) is executed. The region to render defined by the maximum value L2 corresponds to the second region. The region to render defined by the minimum value L1 corresponds to the first region. The minimum value may be preset to a value equivalent to a voxel size. To display the same area when the region to render is varied, the first region is desirably included in the second region.

When imaging objects are blood vessels, the maximum value may be greater than or equal to 2 mm in order to understand connections of the blood vessels. The maximum value may be less than or equal to 10 mm in order not to perform rendering up to a redundant region. The control unit 323 may execute control not to accept input or control to provide an alert when a value not included in these ranges is input as the region to render. The minimum value may be preset to a value equivalent to a voxel size.

In the present embodiment, in order to define a region to render, an example in which the thickness (the length of one side) of a rectangular parallelepiped is input is described; however, any method is applicable as long as a region to render can be defined. For example, when a region to render is a spherical shape, the region to render may be defined by specifying the radius or diameter of a sphere. Alternatively, a region to render may be defined by specifying the number of images (the number of frames) that make up the region to render. Alternatively, a region to render may be defined by specifying the thickness of the region to render in at least one direction.

Maximum intensity projection (MIP), average intensity projection (AIP), and minimum intensity projection (MinIP) are displayed as choices for a rendering method, and MIP is selected in the drawing.

A first position, a center position, and a last position are displayed as choices for a reference position of the region to render, and the center position is selected in the drawing. The thickness of the region to render in the projection direction from an end of medical image data 1000 to the reference position can be input for the reference position, and 7.0 mm is input in the drawing. In other words, the reference position is input such that the center (reference position) of the region to render is located at a position of 7.0 mm from the end of the medical image data 1000 in the projection direction (Z-axis direction). In a process of gradually reducing the region to render (described later), the direction in which the region to render is reduced varies depending on which reference position is selected. When the reference position is the first, the region to render reduces from the last toward the first. When the reference position is the center, the region to render reduces from both the first and the last toward the center. When the reference position is the last, the region to render reduces from the first toward the last.

A user may directly input a period of time (transition time) that is taken to shift the region to render from the maximum value to the minimum value. In the drawing, 3.0 seconds is input as the condition. As for seconds to be input, as a result of the study of the inventor, several seconds are preferable to smoothly proceed with interpretation work, and preferably a period of time shorter than or equal to five seconds is selected. The control unit 323 may execute control not to accept input or control to provide an alert when a value greater than a predetermined threshold as a transition time is input. The predetermined threshold is preferably set to a value shorter than five seconds so as not to interfere with interpretation work. Furthermore, for the efficiency of interpretation work, a value shorter than three seconds is preferably set for the predetermined threshold. A value longer than one second is preferably set for the predetermined threshold so that a change of rendered images can be visually identified. Furthermore, a value longer than two seconds is preferably set for the predetermined threshold so that a change of rendered images can be further tracked and visually identified. Alternatively, not the transition time of the region to render is directly input, but the transition time may be eventually determined by inputting the amount of change per unit time in the region to render. As long as a parameter can determine the transition time of the region to render, the parameter is included in information that represents the transition time of the region to render.

For example, a user can use a mouse to select one of the choices. When a numeric value is directly input, a user can use a keyboard.

The rendering conditions are not limited to those displayed in the display area 1520. Any parameters on rendering may be employed as the rendering conditions. In the present embodiment, as for a method of inputting rendering conditions as well, each rendering condition may be input by any method, such as inputting text to a text box, selecting from a list box, and depressing a button. At least one of the rendering conditions may be set in advance.

A region to render set for the medical image data 1000 according to the present embodiment will be described with reference to FIG. 7. The region to render 1012 indicated by the dashed line is a region to render when the thickness in the Z-axis direction is the maximum value L2. On the other hand, the region to render 1011 indicated by the alternate long and short dashed line is a region to render when the thickness in the Z-axis direction is the minimum value L1. In any region to render, the center position of the region is the same. In other words, in any region to render, the center of the region to render is a reference position. In FIG. 7, the reference position (first, center, or last) of the region to render is defined with reference to the starting point of the Z-axis. Alternatively, the reference position of the region to render may be defined with reference to the end point of the Z-axis. The reference position is not limited to first, center, or last and may be set to a desired position.

A display area 1530 is an area in which thumbnail images of pieces of medical image data 1000, other than the medical image data 1000 acquired in step S1410, are displayed. Medical image data selected by a user from among the thumbnail images displayed in the display area 1530 with the use of the input device 360 may be displayed in the display area 1510. In other words, a rendered image that is displayed in the display area 1510 may be updated with a rendered image of the medical image data selected from among the thumbnail images.

In the case of FIG. 15, a thumbnail image 1531 is selected, and medical image data associated with the thumbnail image 1531 is displayed in the display area 1510. When a user operates a thumbnail image advance icon 1533, thumbnail images that are displayed in the display area 1530 can be sequentially changed.

In S1410 as well, thumbnail images of a plurality of pieces of medical image data 1000 saved in the external storage 310 may be displayed, and medical image data that is used in process of S1430 and subsequent steps may be acquired by selecting one of the thumbnail images.

### S1430: Step of Displaying Rendered Image

In this process, the arithmetic unit 322 generates a rendered image by rendering the medical image data 1000 based on information that indicates the rendering conditions input in step S1420. Here, the region to render (second region) having a center at a position of 7.0 mm in the Z-axis direction from the end of the medical image data 1000 and having a thickness of 10.0 mm in the Z-axis direction is defined. The arithmetic unit 322 generates a rendered image by applying MIP in the region to render in the Z-axis direction set for the projection direction. The control unit 323 outputs the rendered image generated by the arithmetic unit 322 to the display device 350 and causes the display device 350 to display the rendered image. Hereinafter, the MIP image generated when the thickness of the region to render is the maximum value (when the region to render is the second region) is referred to as maximum MIP image.

### S1440: Step of Receiving First Instruction Signal

The user makes an operation for changing the region to render with the use of the input device 360 when the maximum MIP image is being displayed. For example, a right-click of the mouse that serves as the input device 360 may be assigned to an operation for changing the region to render. The control unit 323 receives an instruction signal (first instruction signal) for changing the region to render, which is sent from the input device 360 in response to a user's operation. For example, when the user holds down the right mouse button, the control unit 323 is able to continue receiving the first instruction signal. In this way, a period in which the user continues the operation and the control unit 323 continues receiving the first instruction signal is referred to as a period in which the first instruction signal is being received.

S1450: Step of Displaying Moving image of Rendered images Obtained by Gradually Reducing Region to Render

The arithmetic unit 322 performs rendering while gradually reducing the region to render from the maximum value L2 to the minimum value L1, set in step S1420, in a period in which the control unit 323 is receiving the first instruction signal that the control unit 323 starts receiving in step S1440. The arithmetic unit 322 defines a plurality of regions to render associated with a plurality of thicknesses between the maximum value L2 and the minimum value L1, and generates rendered images respectively associated with the regions to render. The arithmetic unit 322 may sequentially generate a rendered images in order of gradually reducing the thickness of the region to render from the maximum value L2.

The control unit 323 causes the display device 350 to display the plurality of rendered images sequentially generated by the arithmetic unit 322 in order of gradually reducing the thickness of the region to render as a moving image. Thus, the user is able to check a state of gradually shifting from the maximum MIP image from which the overall structure of an imaging object is easy to understand to an image from which the local structure is easy to understand, so a continuous change from the overall structure of an imaging object to the local structure can be understood.

The control unit 323 may determine the thickness of the region to render, which reduces between frames, based on the frame rate of the moving image, the transition time, the maximum value L2, and the minimum value L1, set in S1420. The frame rate may be set to greater than or equal to 10 fps to see a smooth moving image. To see a further smooth moving image, the frame rate may be set to greater than or equal to 30 fps.

### S1460: Step of Detecting End of Reception of First Instruction Signal

The user terminates the operation for changing the region to render when the moving image of the rendered images is being displayed. When the user's operation is stopped, the first instruction signal from the input device 360 stops, and the control unit 323 detects the end of reception of the first instruction signal. For example, when the user stops holding down the right mouse button, the control unit 323 is able to detect the end of reception of the first instruction signal.

### S1470: Step of Terminating Process of Gradually Reducing Region to Render

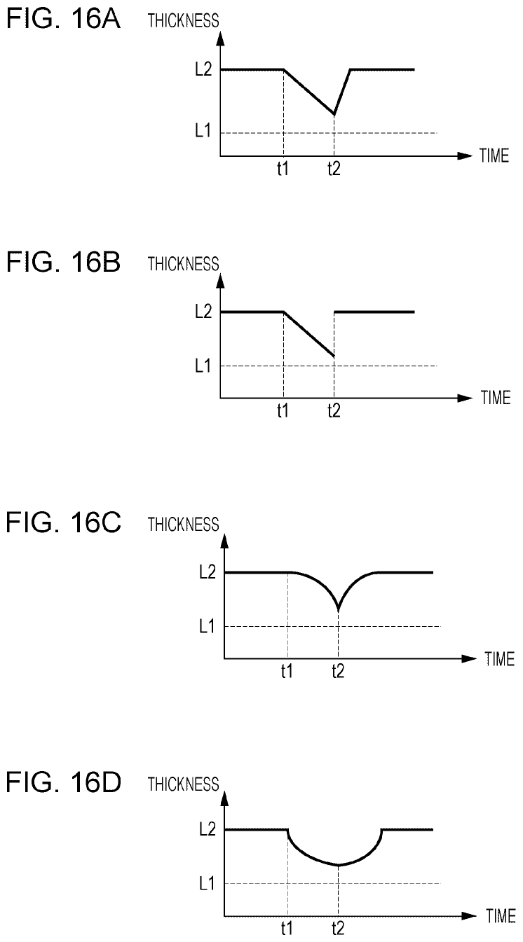

When the control unit 323 detects the end of reception of the first instruction signal, the arithmetic unit 322 terminates the process of performing rendering while gradually reducing the region to render. The rendering process and image display process after the end of reception of the first instruction signal will be described in detail in the description of graphs shown in FIG. 16A to FIG. 16D below.

FIG. 16A to FIG. 16D are graphs that show the relationship between the receiving timing of the first instruction signal and the region to render. The abscissa axis represents time, and the ordinate axis represents the thickness of the region to render. t1 is the timing at which reception of the first instruction signal starts, and t2 is the timing at which reception of the first instruction signal ends. L2 is the maximum value of the thickness of the region to render, set in step 1420. L1 is the minimum value of the thickness of the region to render, set in step 1420.

FIG. 16A is a graph of an example in which the amount of change per unit time in the thickness of the region to render is gradually reduced at a constant rate in a period in which the first instruction signal is being received, and the thickness of the region to render is gradually increased at a constant rate after reception of the first instruction signal ends. In this example, since a change in the thickness of the region to render is constant, the user easily intuitively understands a lapse of time and the amount of change in the region to render. In this example, after reception of the first instruction signal ends, the thickness of the region to render is gradually increased until the region to render reaches the maximum value L2, so a continuous change from the local structure to the overall structure can be understood. Furthermore, in this example, the amount of change per unit time in the thickness of the region to render varies between when the region to render is gradually reduced and when the region to render is gradually increased. In this example, the amount of change per unit time when the region to render is gradually increased is greater than the amount of change per unit time when the region to render is gradually reduced. Thus, when the user wants to check the local structure while checking the overall structure, the user is able to check a change in structure by taking time. After checking of the local structure completes, the screen can be quickly returned to the main image (maximum MIP image) for checking the overall structure, so interpretation work can be efficiently performed.

FIG. 16B is an example in which, when reception of the first instruction signal ends, the maximum MIP image at the time when the thickness of the region to render is the maximum value is displayed. A period in which the first instruction signal is being received in FIG. 16B is similar to FIG. 16A. In this example, after checking of the local structure completes, the focus can be quickly returned to the main image (maximum MIP image) for checking the overall structure, so interpretation work can be efficiently performed.

FIG. 16C is an example in which the amount of change per unit time in the thickness of the region to render varies. In this example, when the thickness of the region to render gradually reduces in a period in which the first instruction signal is being received, the amount of change per unit time gradually increases. Thus, there is no significant change in a rendered image immediately after the user starts the operation, and a change in the rendered image increases with time. Therefore, when the user wants to intensively examine a change in the overall structure, the region to render may be gradually reduced with this method. In this example, when the thickness of the region to render is gradually increased after the end of reception of the first instruction signal, the amount of change per unit time gradually reduces. Therefore, when the user wants to intensively examine a change in the overall structure, the region to render may be gradually increased with this method.

FIG. 16D is an example in which a mode of change in the amount of change per unit time in the thickness of the region to render differs from that of FIG. 16C. In this example, when the thickness of the region to render gradually reduces in a period in which the first instruction signal is being received, the amount of change per unit time gradually reduces. On the other hand, when the thickness of the region to render is gradually increased after the end of reception of the first instruction signal, the amount of change per unit time gradually increases. Therefore, when the user wants to intensively examine a change in the local structure, the region to render may be gradually reduced or gradually increased in this way.

In this way, in all of the examples shown in FIG. 16A to FIG. 16D, reception of the first instruction signal ends before the thickness of the region to render reaches the minimum value L1, so the gradually reducing process terminates in response to the end of reception of the first instruction signal. When the thickness of the region to render reaches the minimum value L1 before reception of the first instruction signal ends, the gradually reducing process may be terminated when the thickness of the region to render reaches the minimum value L1. In other words, the process of gradually reducing the region to render can be terminated when reception of the first instruction signal ends or when the region to render reaches the minimum value L1 (first region).

As long as the region to render gradually reduces in a period in which the first instruction signal is being received, the region to render may be changed with any method. Any combination of one of the controls over the region to render in a period in which the first instruction signal is being received, described in FIG. 16A to FIG. 16D, and one of the controls over the region to render after reception of the first instruction signal ends is applicable.

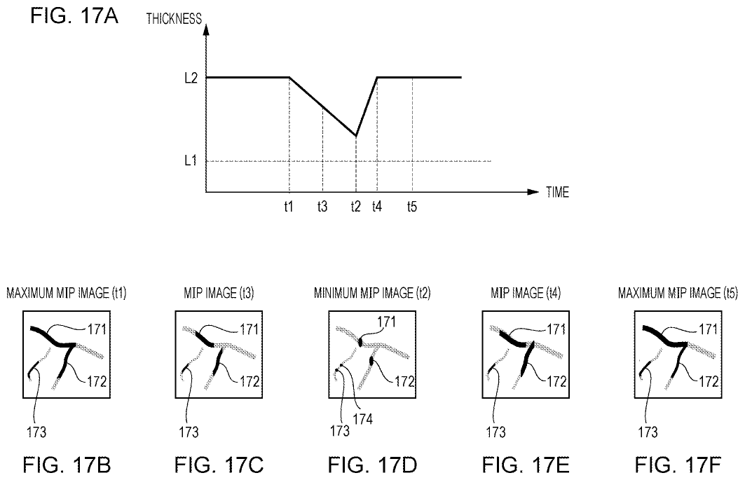

FIG. 17B to FIG. 17F show changes of a rendered image when the region to render is changed in accordance with the sequence shown in FIG. 17A. For the sake of convenience, in order to easily understand the positional relationship among blood vessels, the rendered images of the blood vessels projected in all the region in the Z-axis direction are displayed in gray color in a superposition manner; however, these are not displayed in the actual image.

FIG. 17B is a rendered image (maximum MIP image) when the thickness of the region to render before time ti is the maximum value L2. As shown in FIG. 17B, blood vessels 171, 172, 173 are displayed with continuity on the maximum MIP image, and blood vessel running, or the like, can be understood. FIG. 17C is a rendered image of the region to render associated with time t3 after time t1 and before the thickness of the region to render reaches the minimum value L1. FIG. 17D is a rendered image (minimum MIP image) of the region to render (minimum value L1) associated with time t2. Here, the MIP image generated at the time when the thickness of the region to render is the minimum value (when the region to render is the first region) is referred to as minimum MIP image. In the minimum MIP image shown in FIG. 17D, the image of a blood vessel 174 that does not appear in the MIP image shown in FIG. 17B or the MIP image shown in FIG. 17C is displayed. The blood vessel 174 overlaps the blood vessel 173 in the Z-axis direction, and is not shown when subjected to maximum intensity projection.

In this way, by gradually reducing the region to render in a period in which the first instruction signal is being received, a continuous change from the overall structure to the local structure can be understood. In other words, while a user tracks the running paths of blood vessels, the user can understand the local structure of the blood vessels being interpreted.

FIG. 17E is a rendered image of the region to render associated with time t4 after time t2 and before the thickness of the region to render reaches the maximum value L2. FIG. 17F is a rendered image that is displayed after the thickness of the region to render reaches the maximum value L2. The rendered image shown in FIG. 17F is similar to the rendered image shown in FIG. 17B and is the maximum MIP image.

In this way, when reception of the first instruction signal ends, the region to render gradually increases, and a continuous change from the overall structure to the local structure can be understood. In other words, while a user tracks the running paths of blood vessels, the user can shift the image to the maximum MIP image that is the main image.

In the present embodiment, the image display method based on photoacoustic image data that is volume data from photoacoustic waves is described. The image display method according to the present embodiment may also be applied to volume data other than photoacoustic image data. The image display method according to the present embodiment may be applied to volume data obtained by a modality, such as an ultrasonic diagnostic system, an MRI system, an X-ray CT system, and a PET system. Particularly, the image display method according to the present embodiment may be applied to volume data including image data that represents blood vessels. Blood vessels have complex structures, and how blood vessels are running ahead cannot be estimated from a cross-sectional image. When a wide region is rendered, positional relationship among complex blood vessels cannot be understood. Therefore, the image display method according to the present embodiment may be applied to volume data including image data that represents blood vessels. For example, at least one of photoacoustic image data, magnetic resonance angiography (MRA) image data, X-ray computed tomography angiography (CTA) image data, and Doppler image data may be applied as volume data including image data that represents blood vessels.

In the present embodiment, the example in which a rendered image using medical image data of a single image type is displayed is described. Alternatively, a rendered image using medical image data of multiple image types may be displayed. For example, a rendered image generated using medical image data of an image type may be set as a base image, a rendered image may be generated with the method described in the present embodiment by using medical image data of a different image type, and the rendered image may be superimposed on the base image. In other words, a composite image obtained by combining an additional rendered image based on additional medical image data with medical image data subjected to rendering of the present embodiment may be generated and displayed. Not only a superimposed image but also a parallel image, or the like, may be employed as a composite image. In a superimposed image, a rendered image that is a base image may function as a reference image when the region to render is fixed. For example, a rendered image of MRI image data or ultrasonic image data including a tumor image may be set as a base image, rendering of the present embodiment may be applied to photoacoustic image data in which blood vessels are drawn, and a rendered image of which the region to render changes may be superimposed on the base image. Thus, since the tumor image that appears in the base image is fixed, the positional relationship between a tumor and blood vessels around the tumor can be easily understood.

### Fifth Embodiment

In the present embodiment, the case where the region to render reaches the minimum value before reception of the first instruction signal (signal based on a gradually reducing instruction from a user) ends will be described. In the present embodiment, description will be made by using a similar apparatus to those of the first to fourth embodiments, like reference numerals denote similar components, and the detailed description thereof is omitted.

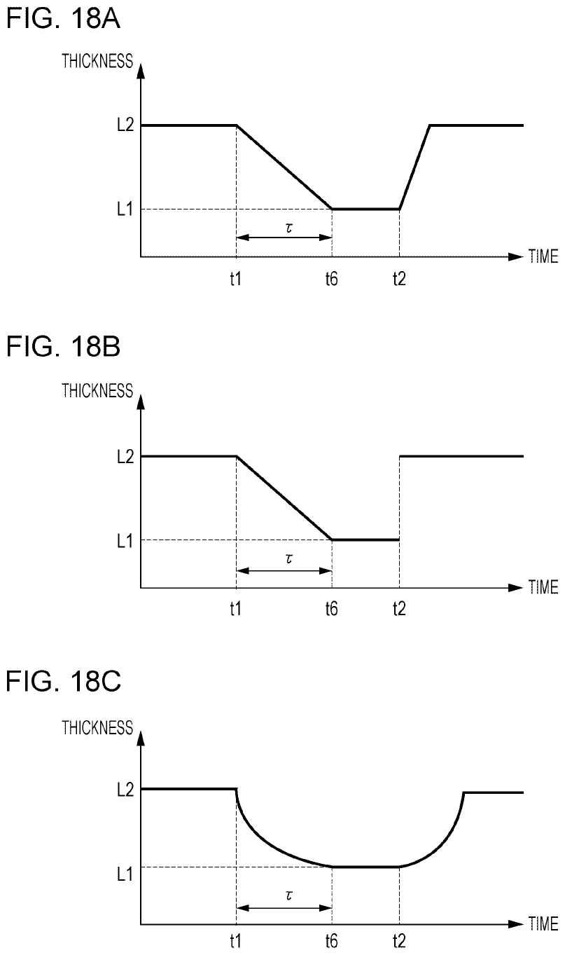

FIG. 18A to FIG. 18C are graphs that show the relationship between the receiving timing of an instruction signal and the region to render according to the present embodiment. The abscissa axis represents time, and the ordinate axis represents the thickness of the region to render. t1 is the timing at which reception of the first instruction signal starts, and t2 is the timing at which reception of the first instruction signal ends. T is a transition time set in step S1420, and t6 is the timing at which the transition time T (predetermined period) elapses from the start of reception of the first instruction signal. L2 is the maximum value of the thickness of the region to render, set in step S1420. L1 is the minimum value of the thickness of the region to render, set in step S1420.

FIG. 18A to FIG. 18C are examples when a user continues an operation for gradually reducing the thickness of the region to render even after the thickness reaches the minimum value L1. In this example, the process of gradually reducing the region to render is terminated at time t6 at which the thickness of the region to render reaches the minimum value L1. In other words, the process of gradually reducing the region to render is executed such that the thickness of the region to render becomes the minimum value L1 (first region) when the transition time T elapses from the start of reception of the first instruction signal. Subsequently, the minimum MIP image is continuously displayed after the thickness of the region to render reaches the minimum value L1 and in a period in which the first instruction signal is being received.

In FIG. 18A, when reception of the first instruction signal ends while the minimum MIP image is being displayed, the thickness of the region to render is gradually increased to the maximum value L2. In FIG. 18B, when reception of the first instruction signal ends while the minimum MIP image is being displayed, display is switched from the minimum MIP image to the maximum MIP image. In FIG. 18C, when reception of the first instruction signal ends while the minimum MIP image is being displayed, the thickness of the region to render is gradually increased to the maximum value L2. In FIG. 18C, the thickness of the region to render is gradually increased to the maximum value L2 while the amount of change in the region to render is gradually increased.

In this way, while a user is continuously making an operation for checking the local structure, the minimum MIP image is displayed. Thus, the user sufficiently understands the local structure with an easy operation, and the user is also able to smoothly shift into an image representing the overall structure.

In the present embodiment, the example in which, when the first instruction signal is being received at the time when the transition time τ elapses from the start of reception of the first instruction signal, display of the minimum MIP image is continued is described. However, control over the region to render at the time when the transition time τ elapses from the start of reception of the first instruction signal is not limited to this example. For example, when the transition time τ, has elapsed from the start of reception of the first instruction signal, the region to render may be gradually increased to the maximum value L2 regardless of whether the first instruction signal is being received. Also, when the transition time τ elapses from the start of reception of the first instruction signal, display may be switched from the minimum MIP image to the maximum MIP image regardless of whether the first instruction signal is being received.

In the present embodiment, the example in which the region to render is gradually reduced when a user operates the specific operation is described; however, the timing of starting the gradually reducing process is not limited thereto. For example, the user may perform an operation for changing the reference position of the maximum MIP image (image advance operation), and, while the maximum MIP image is being displayed through image advance, the gradually reducing process may be started when a predetermined period elapses from when the image advance operation is terminated. In other words, the information processing apparatus 320 may start the gradually reducing process when a predetermined period elapses from the end of reception of an instruction signal based on image advance operation. In this case, when the transition time τ elapses from the start of the gradually reducing process, the gradually reducing process may be terminated. An operation for scrolling the wheel of the mouse that serves as the input device 360 may be assigned to an image advance operation.

In this way, a user searches for a region that the user wants to understand by image advance and, when the user stops the image advance operation at the time when the user finds the desired region in the maximum MIP image, display can be shifted into a moving image of rendered images for understanding the local structure. A period that is taken from the end of image advance to a transition to the gradually reducing process may be set in advance or may be specified by a user with the use of the input device 360.

### Sixth Embodiment

In the present embodiment, the case where a user makes an operation for gradually reducing the region to render after reception of the first instruction signal ends or after the region to render reaches the minimum value and in a period in which the process of gradually increasing the region to render is being executed will be described. When the user makes the operation and the information processing apparatus 320 receives a second instruction signal in response to this operation while the region to render is being gradually increased, the process of gradually reducing the region to render is executed again. In the present embodiment, description will be made by using a similar apparatus to those of the first to fifth embodiments, like reference numerals denote similar components, and the detailed description thereof is omitted.

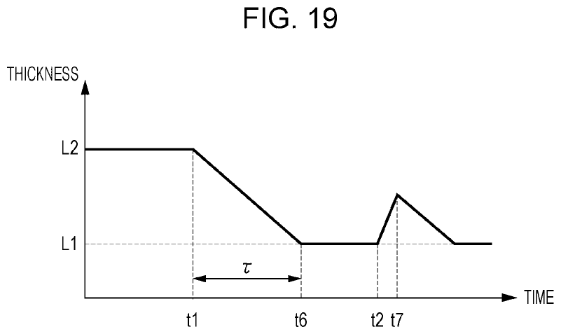

FIG. 19 is a graph that shows the relationship between the receiving timing of an instruction signal and the region to render according to the present embodiment. The abscissa axis represents time, and the ordinate axis represents the thickness of the region to render. t1 is the timing at which reception of the first instruction signal starts, and t2 is the timing at which reception of the first instruction signal ends. τ is a transition time set in step S1420, and t6 is the timing at which the transition time τ (predetermined period) elapses from the start of reception of the first instruction signal. t7 is the timing at which reception of the second instruction signal starts in response to an operation from the user after time t2. L2 is the maximum value of the thickness of the region to render, set in step S1420. L1 is the minimum value of the thickness of the region to render, set in step S1420.

FIG. 19 is an example of the case where the user makes an operation for gradually reducing the thickness again after reception of the first instruction signal ends and when the thickness of the region to render is being gradually increased. In this example, at time t7 at which the user makes the operation again, the process of gradually increasing the region to render is terminated. Then, in a period in which the user is continuously making the operation again (that is, a period in which the second instruction signal is being received), the region to render is gradually reduced to the minimum value L1. At this time, if the period that is taken to reach the minimum value L1 is determined to the transition time τ, the amount of change per unit time in the region to render varies for each operation. Therefore, the amount of change set in the first gradually reducing process may be set for the amount of change per unit time in the region to render at the time of executing the second gradually reducing process. Thus, when the gradually reducing process is executed multiple times through multiple operations, the user can check a moving image of rendered images of which the region to render gradually reduces without a feeling of strangeness between operations.

When the user makes an operation for gradually reducing the thickness while the process of gradually increasing the region to render is being executed after reception of the first instruction signal ends before a lapse of the transition time τ from time t1 as well, the gradually reducing process can be repeated. In other words, when the region to render falls between the maximum value L2 and the minimum value L1 as well, the gradually reducing process and the gradually increasing process can be repeatedly executed depending on whether the first instruction signal is continuously received.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus comprising:
an image data acquisition unit configured to acquire three-dimensional medical image data; and
a display control unit configured to cause a display unit to display a rendered image by rendering the three-dimensional medical image data in a region to render,
wherein
the display control unit is configured to cause the display unit to display the rendered image by rendering the three-dimensional medical image data in a first region set for the region to render,
the display control unit is configured to cause the display unit to display a moving image of the rendered images respectively associated with a plurality of the regions to render different from each other by gradually increasing the region to render from the first region to a second region in a period in which the display control unit is receiving a first instruction signal that is sent in response to an instruction from a user, and
the display control unit is configured to terminate a process of gradually increasing the region to render when the display control unit stops receiving the first instruction signal or when the region to render reaches the second region.

2. The information processing apparatus according to claim 1, wherein the display control unit is configured to, when the display control unit stops receiving the first instruction signal, cause the display unit to display a moving image of the rendered images respectively associated with a plurality of the regions to render different from each other by gradually reducing the region to render of the rendered image, which is displayed when the display control unit stops receiving the first instruction signal, until the region to render reaches the first region.

3. The information processing apparatus according to claim 2, wherein the display control unit is configured to, in a period in which the display control unit is receiving a second instruction signal that is sent in response to an instruction from the user while the display control unit is gradually reducing the region to render, cause the display unit to display a moving image of the rendered images respectively associated with a plurality of the regions to render different from each other by gradually increasing the region to render of the rendered image, which is displayed when the display control unit receives the second instruction signal, until the region to render reaches the second region.

4. The information processing apparatus according to claim 2, wherein the display control unit is configured to control the region to render such that an amount of chance per unit time in the region to render when the region to render is gradually reduced is different from an amount of change per unit time in the region to render when the region to render is gradually increased.

5. The information processing apparatus according to claim 4, wherein the display control unit is configured to control the region to render such that a period of time that is taken to change the region to render from the first region to the second region is shorter than a period of time that is taken to change the region to render from the second region to the first region.

6. The information processing apparatus according to claim 1, wherein the display control unit is configured to, when the display control unit stops receiving the first instruction signal, cause the display unit to display the rendered image in the first region set for the region to render.

7. The information processing apparatus according to claim 1, wherein the display control unit is configured to, when the display control unit stops receiving the first instruction signal, cause the display unit to display the rendered image that is displayed when the display control unit stops receiving the first instruction signal.

8. The information processing apparatus according to claim 1, wherein
the display control unit is configured to, when a predetermined period elapses from when the display control unit starts receiving the first instruction signal, gradually increase the region to render such that the region to render becomes the second region, and
the display control unit is configured to, in the period in which the display control unit is receiving the first instruction signal and after the predetermined period elapses from when the display control unit starts receiving the first instruction signal, cause the display unit to display the rendered image in the second region set for the region to render.

9. The information processing apparatus according to claim 1, wherein the display control unit is configured to, in the period in which the display control unit is receiving the first instruction signal, keep an amount of change per unit time in the region to render constant.

10. The information processing apparatus according to claim 1, wherein the display control unit is configured to, in the period in which the display control unit is receiving the first instruction signal, gradually reduce an amount of change per unit time in the region to render.

11. The information processing apparatus according to claim 1, wherein the display control unit is configured to cause the display unit to display the rendered image by rendering the three-dimensional medical image data in the region to render through any one of maximum intensity projection, average intensity projection, minimum intensity projection, surface rendering, and volume rendering.

12. The information processing apparatus according to claim 1, wherein
the image data acquisition unit is configured to acquire additional three-dimensional medical image data of a different in image type from the three-dimensional medical image data,
the display control unit is configured to generate an additional rendered image by rendering the additional three-dimensional medical image data in the first region set for the region to render, and
the display control unit is configured to cause the display unit to display a composite image of the rendered image of the three-dimensional medical image data and the additional rendered image.

13. The information processing apparatus according to claim 1, wherein the three-dimensional medical image data is medical image data generated by a modality that is any one of a photoacoustic imaging system, an ultrasonic diagnostic system, a magnetic resonance imaging system, an X-ray computed tomography system, and a positron emission tomography system.

14. The information processing apparatus according to claim 1, wherein the first region is included in the second region.

15. The information processing apparatus according to claim 1, wherein the display control unit is configured to, in a period in which the display control unit is receiving the first instruction signal, gradually increase a thickness of the region to render in at least one direction.

16. An information processing method comprising:
acquiring three-dimensional medical image data;
causing a display unit to display a rendered image by rendering the three-dimensional medical image data in a first region set for a region to render;
causing the display unit to display a moving image of the rendered images respectively associated with a plurality of the regions to render different from each other by gradually increasing the region to render from the first region to a second region in a period in which the display control unit is receiving a first instruction signal that is sent in response to an instruction from a user; and
terminating a process of gradually increasing the region to render when the display control unit stops receiving the first instruction signal or when the region to render reaches the second region.

17. A non-transitory computer-readable storage medium storing a program for causing a computer to execute the information processing method according to claim 16.
